(19) United States
(12) Patent Application Publication
(10) Pub. No.: US 2007/0191844 A1
(43) Pub. Date: Aug. 16, 2007

(54) In-series, dual locking mechanism device

(75) Inventors: Thomas A. Carls, Memphis, TN (US); Jonathan M. Dewey, Memphis, TN (US)

Correspondence Address: HESLIN ROTHENBERG FARLEY & MESITI P.C., 5 COLUMBIA CIRCLE, ALBANY, NY, 12203, US

(73) Assignee: SDGI Holdings, Inc., Wilmington, DE (US)

(21) Appl. No.: 11/343,568

(22) Filed: Jan. 31, 2006

(51) Int. Cl. A61F 2/30

(52) U.S. Cl. 606/61

(57) ABSTRACT

An in-series, dual locking mechanism device for securing at least one implant having a clamp, a locking mechanism and a bone fastener. The clamp is configured to include a channel for receiving the implant and a locking mechanism. The locking mechanism is structured to secure the implant within the clamp by applying a force in at least two locations that are configured to be adjacent and in-series relative to each other. The locking mechanism functions to reduce the resultant internal stresses realized by the implant that may lead to implant fracture or fatigue. The implant is generally continuous and is preferably rod shaped. The shape of the implant thereby allowing it to be utilized in various surgical procedural applications. The clamp is further configured for the attachment of a bone fastener. The bone fastener functioning to rigidly fix the clamp to the bone.

### TECHNICAL FIELD

This invention relates generally to implantable, surgical devices and, in particular, to an improved locking mechanism device resulting in the reduction of internal stresses to an implant.

### BACKGROUND OF INVENTION

Post-operatively, implanted medical devices may fail by sudden fracture or cyclic fatigue of one of the components that comprise the medical device. Failures of medical devices in vivo may be caused by numerous possible events or combination of events. These events may include, improper sizing of device, incorrect alignment of the device, a manufacturing defect within the device, improper materials being used to manufacture the device, use of the device in a contraindicative clinical setting, improper device design, resultant in vivo forces being applied that exceed the design limitations of the device, the device being subject to abnormal motion patterns and loss of device integrity.

Decreasing resultant stresses realized within the components of a medical device post-operatively is critical to reducing the possibility of latent failures when certain construct materials are used. Currently, many locking mechanisms induce failure within an implant because of high stress concentrations resulting from single point loading configurations. The invention described herein addresses the failure modality that is seen at single point component securement interfaces by distributing a locking force over a larger implant contact area and thereby reducing the stress concentrations seen within an implant.

### SUMMARY OF THE INVENTION

The present invention provides an in-series, dual locking mechanism device for use in securing implants within an orthopaedic device. The purpose of the invention is to provide a mechanism for locking an implant in a fixed position in a manner such that the internal stresses realized by the implant are reduced. It is understood that the drawings and specific language used herein is for the purposes of promoting and understanding of the principles of the invention and that no limitation of the scope of the invention is thereby intended. Any alteration or modification to the drawings illustrated, and further application of the principles of the invention as illustrated herein are contemplated as would normally occur to one skilled in the art to which the invention relates.

In meeting this design objective, the in-series, dual locking mechanism device may be comprised of a clamp that may include a channel in which an elongate and continuous implant is placed. The clamp may be further configured to include a locking mechanism that secures the placed implant in a fixed location. The locking mechanism being operable between an open position wherein the implant is free to move and a closed position wherein the implant is rigidly fixed in the clamp. In the closed position, the locking mechanism is structured to apply a pressing force to the implant in at least two adjacent and in-series locations. The clamp is configured to be attached to a bone by a bone fastener that is fixed to and projects from the clamp.

The channel within the clamp is typically U-shaped, defined by a floor and at least two parallel side walls that project in an upward direction from the floor. The clamp may be configured in an alternative form including, but not limited to a cylinder or a bracket. Located on the side walls' inner surfaces may be threads or an internal cam surface. The locking mechanism is preferably comprised of at least two locking caps that maybe constructed with external threads or a corresponding external cam surface. When in the closed position, the locking caps are inserted into the threads or the internal cam mechanism located on the side walls of the channel and are rotated until making contact with the inserted implant that lies on the floor of the channel, thereby holding the implant in position.

The clamp can be configured to be joined with a bone fastener. Preferably, the bone fastener is inserted into a through hole in the floor of the channel, though alternatively, the bone fastener may be integrally attached to the clamp. The bone fastener may then project from the clamp engaging a bone. Depending upon the bone location, alternative bone fasteners may be used. These alternative bone fasteners include, but are not limited to pedicle bone screws, bone fixation posts, staples, hooks, anchors, fixed head screws, moveable head screws and collared dual threaded combination post/screws.

The in-series, dual locking mechanism device rigidly secures an implant in a fixed position within a channel. Preferably, the implant is shaped as an elongate and continuous rod. Alternative implants may be utilized in the device including, but not limited to plates, bars, tethers, cables, elastic structures and dynamic stabilization members. The material comprising the implant is preferably selected from the group consisting of stainless steel, carbon fiber composite, titanium, cobalt-chromium, shape memory metal, resorbable polymers, bio-inert metal, bio-inert polymeric materials, and any combinations of these materials.

The preferred embodiment of the in-series, dual locking mechanism device is used to secure an implant to a bone. Typically, the device is comprised of a clamp that includes at least one channel in which an implant is placed, a locking mechanism that is preferably integrally associated with the clamp and secures the implant, and a bone fastener that is attached to the clamp. The method of using the in-series, dual locking mechanism device can include the steps of: 1) selecting the preferred bone fastener from the kit and attaching it to the clamp; 2) drilling a pilot hole into at least one bone; 3) inserting the bone fastener into the pilot hole and fixing the clamp to the outer surface of the bone; 4) placing the implant within the channel; and 5) locking the implant into the clamp by engaging the locking mechanism into the closed position.

### BRIEF DESCRIPTION OF THE DRAWINGS

The subject matter which is regarded as the invention is particularly pointed out and distinctly claimed in the claims at the conclusion of the specification. The features and advantages of the invention will be apparent from the following detailed description taken in conjunction with the accompanying drawings, which drawings illustrate several embodiments of the invention.

FIG. 1 is a perspective view of the device;

FIG. 2 is a perspective view of the clamp;

FIG. 3 is a top plan view of the device;

FIG. 4 is a perspective view of an alternative embodiment of the device;

FIG. 5 is an exploded view of the device of FIG. 4;

FIG. 6 is a top plan view of the device of FIG. 4;

FIG. 7 is a perspective view of another alternative embodiment of the device;

FIG. 8 is an exploded view of the device of FIG. 7;

FIG. 9 is a side elevation of the device of FIG. 7 before closing the locking mechanism;

FIG. 10 is a side elevation of the device of FIG. 7 after closing the locking mechanism;

FIG. 11 is a top plan view of the device of FIG. 7;

FIG. 12 is a sectional view along line 12-12 of the device of FIG. 7;

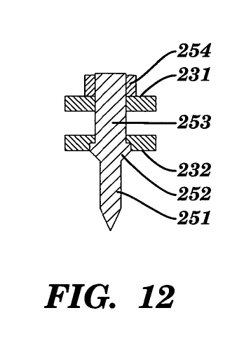

FIG. 13 is an exploded view of the collared bone fastener and locking nut;

FIG. 14 is a perspective view of the clamp;

FIG. 15 is a perspective view of another alternative embodiment of the device;

FIG. 16 is a top plan view of the device of FIG. 15; and

FIG. 17 is a top plan view of the device of FIG. 15.

### DETAILED DESCRIPTION OF THE INVENTION

Locking mechanism devices are utilized to secure implants in a fixed position following final alignment placement. The in-series, dual locking mechanism device includes a clamp wherein the implant is placed, a locking mechanism for securing the implant and a bone fastener for securing the clamp to a bone. Each of the devices described include a locking mechanism that is constructed to engage and fixate the implant in two locations that are adjacent or in relative close proximity to each other and are arranged in a straight line or in-series with no offset deviation relative to each other. The in-series, dual locking mechanism device reduces the internal stresses realized in the implant when secured by distributing the applied locking force over a broader length or area of the implant, thereby decreasing the possibility of implant failure post-operatively.

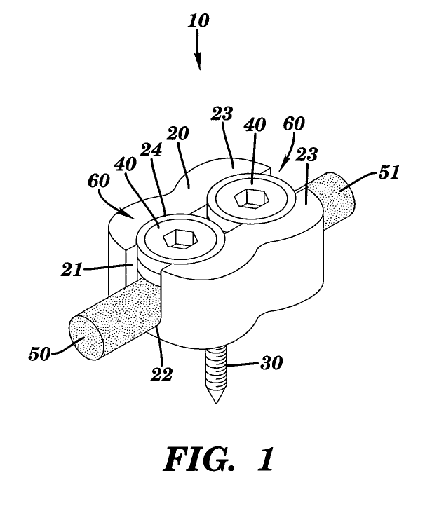

FIG. 1 shows the general arrangement of a preferred embodiment of the in-series, dual locking mechanism device 10. Generally, the in-series, dual locking mechanism device 10 includes a clamp 20, a bone fastener 30, a channel 21, an implant 50, and a locking mechanism 60.

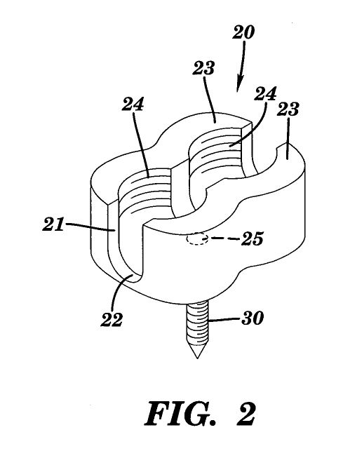

With reference to FIGS. 1-3, the in-series, dual locking mechanism device 10 is comprised of a clamp 20 wherein a channel 21 is preferably defined by a floor member 22 and a pair of wall members 23. The wall members 23 being preferably parallel relative to each other and project in an upward manner from the floor member 22. The wall members 23 together with the floor member 22 form a U-shaped channel 21 approximately sized to receive an implant 50. The internal sides of the wall members 23 may include internal threads 24 or alternatively an internal cam surface (Not Shown) to preferably engage a locking cap 40. The wall members 23 preferably include at least two sets of internal threads 24 or internal cam surfaces (Not Shown) arranged in-line, substantially along the longitudinal axis of the channel 21. As seen in FIG. 1, the two sets of internal threads 24 or alternatively internal cam surfaces (Not Shown) may be positioned in close proximity to each other, substantially along the longitudinal axis of the channel 21. The orientation of the locking mechanism 60 described herein allows for the locking force to be applied over greater or broader area of the implant, thereby resulting in less actual stress to the implant. If the spacing of the locking force is too distant, the benefits of the in-series, dual locking mechanism will not be achieved. Typically as shown in FIG. 2, at least one through hole 25 is located in the floor member 22. The hole 25 typically receives a bone fastener 30 prior to the insertion of an implant 50. The longitudinal axis of the bone fastener 30 may be at a fixed angle relative to the floor member 22 following insertion into the hole 25 or be allowed to pivot within the hole 25. The hole 25 may be counter bored, counter sunk, slotted, have a spherical seat, keyed or any combination or derivation of these manufacturing techniques, to allow the top portion of the bone fastener 30 to sit below the surface of the floor member 22. The bone fastener 30 is typically configured as a bone screw (Not Shown) though, alternative bone fasteners may be utilized including, but not limited to bone fixation posts (Not Shown), bone staples (Not Shown), hooks (Not Shown), anchors (Not Shown), fixed head screws (Not Shown) and moveable head screws (Not Shown). It is understood to those skilled in the art that the bone fastener attachment structure described is for example only and that other configurations may be used, including a clamp 20 configured to be integrally coupled to a bone fastener 30.

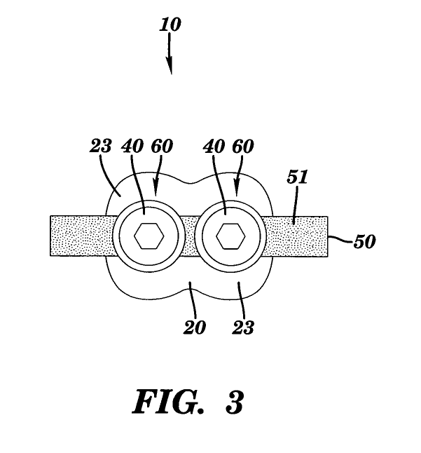

As shown in FIGS. 1 and 3, the locking mechanism 60 is illustrated as including at least two externally threaded locking caps 40 threadably engaged with the internal threads 24 of the wall members 23, although it is understood to those skilled in the art that other configurations are contemplated, including a locking cap configured to include an external cam surface (Not Shown) that engages with an internal cam surface (Not Shown) located within each wall member 23. In the open position, the threaded locking caps 40 of the locking mechanism 60 initiate engagement with the internal threads 24 of the wall members 23 and allow the implant 50 to move freely within the channel 21. When in the closed position, preferably the threaded locking caps 40 of the locking mechanism 60, are substantially engaged with the internal threads 24 usually resulting in a pressing engagement or a compressive force being applied to the top surface 51 of the implant 50. As shown in FIG. 3, the implant 50 is locked by at least two locking caps 40 preferably positioned adjacent and in-series to each other, substantially along the longitudinal axis of the channel 21.

Preferably, the implant 50 secured by the in-series, dual locking mechanism device 10 is shaped as an elongate and continuous rod. Alternative implants 50 may be secured by the in-series, dual locking mechanism device 10, these include, but are not limited to plates, bars, tethers, cables, elastic structures and dynamic stabilization members. The implant 50 may be comprised of a material selected from the group consisting of stainless steel, carbon fiber composite, titanium, cobalt-chromium, shape memory metal, resorbable polymers, bio-inert metal, bio-inert polymeric materials, and combinations of these materials.

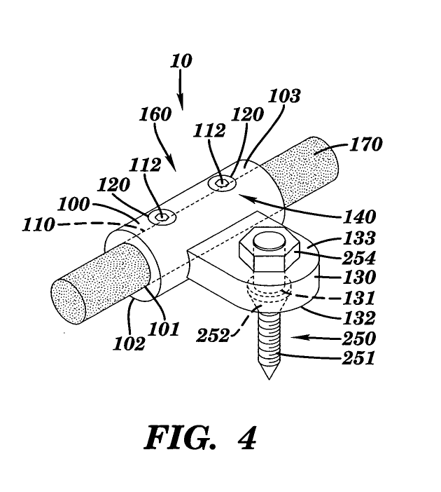

Referring to FIGS. 4-6, an in-series, dual locking mechanism device 10 in accordance with another embodiment is shown and preferably includes a clamp 160 comprised of a cylinder 100 through which a channel 110 passes, a locking mechanism 140 which typically includes at least two locking caps 112, an offset flange 130, which may include at least one through hole 131 for receiving a bone fastener 250.

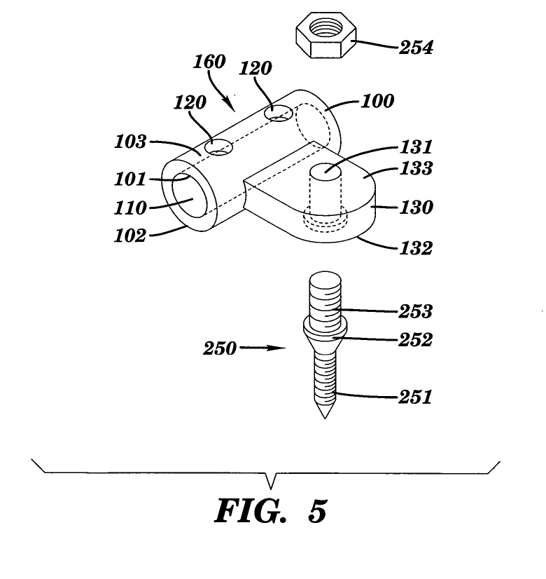

As seen in FIG. 5, the cylinder 100 includes an inner diameter 101 and an outer diameter 102. The cylinder generally has a top, external surface 103 located on the outer diameter 102. The inner diameter 101 is preferably configured and dimensioned to receive an implant 170. Preferably, the implant 170 secured within the cylinder 100 is shaped as an elongate and continuous rod. Alternative implants 170 may be secured within the cylinder 100, these include, but are not limited to plates, bars, tethers, cables, elastic structures and dynamic stabilization members. Further, the implant 170 may be comprised of a material selected from the group consisting of stainless steel, carbon fiber composite, titanium, cobalt-chromium, shape memory metal, resorbable polymers, bio-inert metal, bio-inert polymeric materials, and combinations of these materials.

Referring to FIGS. 4 and 5 the cylinder 100 may include at least two holes 120 which preferably pass from the top, external surface 103 through the outer diameter 102 and project into the inner diameter 101. The centerline of the holes 120 may be about normal to the top, external surface 103, though it is contemplated that the centerline of the holes 120 may be angled relative to the top external surface 103. For all hole orientations, the location of the holes' 120 exit points may be arranged in-series, substantially along the longitudinal axis of the cylinder 100. The exit points of the holes 120 are also preferably positioned adjacent to each other, substantially along the longitudinal axis of the cylinder 100.

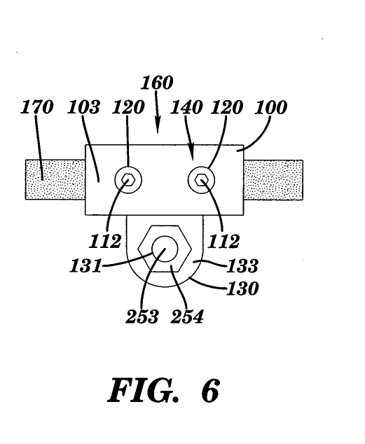

The locking mechanism 140 is illustrated in FIG. 6 to include at least two externally threaded locking caps 112 whereby the locking caps 112 preferably engage the internal threads of the holes 120, although other configurations are contemplated including a locking cap configured to include an external cam surface (Not Shown) that engages with an internal cam surface located within the holes 120. In the open position, the locking caps 112 of the locking mechanism 140 are inserted and threadably engage the holes 120, but do not project into the channel 110 thereby allowing the implant 170 to move freely within the channel 110 over the length of the cylinder 100. The closed position of the locking mechanism 140 is typically achieved when the locking caps 112 protrude into the channel 110 and pressingly engage the implant 170. The implant 170 is fixed by at least two locking caps 112 with preferably contact points that are adjacent and in-series to each other, substantially along the longitudinal axis of the cylinder 100.

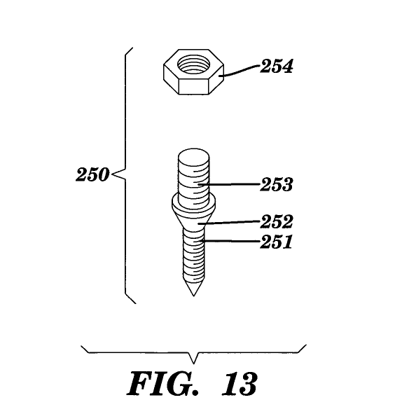

As shown in FIGS. 4-6, the in-series, dual locking mechanism device 10 is preferably secured to a bone by a bone fastener 250 that passes through the offset flange 130, though it is contemplated and understood by those skilled in the art that the bone fastener 250 may be integrally attached to the bottom surface 132. The offset flange 130 is typically fixed at an almost perpendicular angle to the side of the cylinder 100. At least one through hole 131 for receiving a bone fastener 250 is located within the offset flange 130. Following insertion into the offset flange 130, the longitudinal axis of the bone fastener 250 is oriented approximately normal thereto, or alternatively the bone fastener 250 may be allowed to pivot within the hole 131 allowing for angulation of the bone fastener 250. The bone fastener 250 may be configured as a bone screw (Not Shown), but alternative configurations of bone fasteners may include, but are not limited to bone fixation posts (Not Shown), bone staples (Not Shown), hooks (Not Shown), anchors (Not Shown), fixed head screws (Not Shown) and moveable head screws (Not Shown). The embodiment of the bone fastener 250 as seen at FIG. 13, is a collared bone fastener 250 that includes two separate sets of threads. One portion of the collared bone fastener 250 is comprised of external threads 251 that are configured to self-thread into a bone. Attached at the end of the external threads 251 portion is a fixed collar 252. A threaded post 253 portion is fixed to the opposite side of the fixed collar 252 relative to the external thread 251 portion. As seen in FIG. 5, the collared bone fastener 250 may be inserted into the offset flange 130 from the direction of the bottom surface 132. Preferably, the collar 252 would make contact with bottom surface 132 with the threaded post 253 projecting above the top surface 133. The in-series, dual locking mechanism device 10 being preferably secured to the collared bone fastener 250 with a locking nut 254.

It is understood to those skilled in the art that the bone fastener 30, 250 may be attached to embodiments described above in an alternative configuration. For example, the clamp 20 may be structured to include an offset flange 130 to which the bone fastener 30 attaches. Further, the cylinder 100 may be configured to allow for the bone fastener 30 to be attached to the bottom, external surface (Not Shown) of the cylinder 100 either in a rigid or modular fashion.

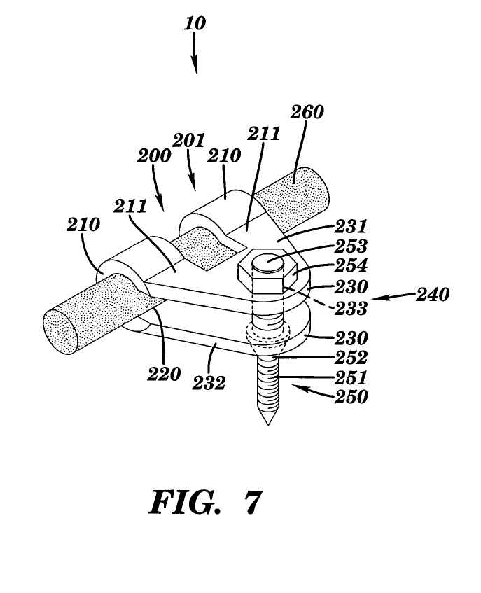

Another embodiment of the in-series, dual locking mechanism device 10 shown in FIGS. 7-11 requires the clamp to be preferably manufactured from a material that has a springy or resiliant/elastic-like material property, in that when loaded or deformed, the material will inherently try to regain its original shape. This further embodiment preferably includes a clamp 200 which is generally in the form of a bracket 201. The clamp is preferably comprised of least one pair of loops 210 that create a channel 220 through which the implant 260 may pass. Each loop 210 typically has an attached pair of legs 211 whereby each pair of legs 211 preferably are joined together by a tab 230. The tab 230 may include at least one through hole 233 for preferably receiving a collared bone fastener 250.

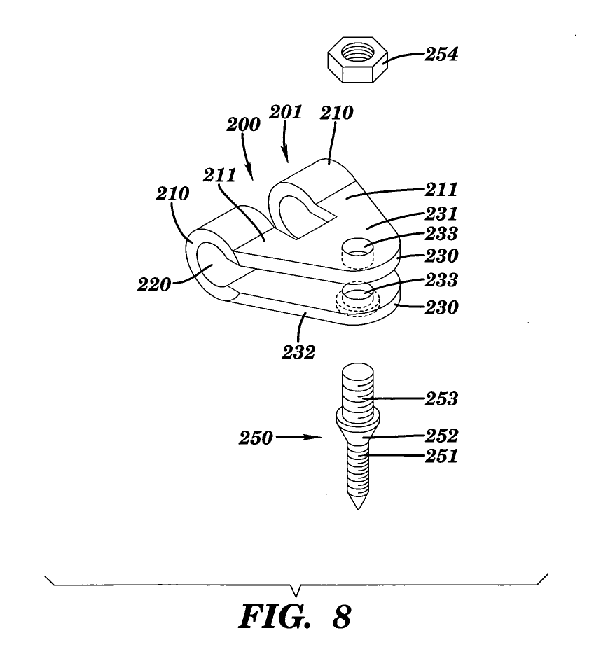

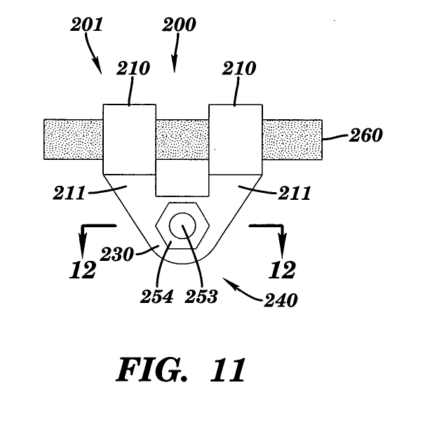

As seen in FIG. 8, the channel 220 for receiving the implant 260 is formed by at least one pair of loops 210. Referring to FIGS. 7 and 11, the pair of loops 210 may be oriented along the longitudinal axis of the implant 260 in an in-series and preferably adjacent position. Preferably, the implant 260 secured within the channel 220 is shaped as an elongate and continuous rod. Alternative implants 260 may be secured within the channel 220, these include, but are not limited to plates, bars, tethers, cables, elastic structures and dynamic stabilization members. Further, the implant 260 may be comprised of a material selected from the group consisting of stainless steel, carbon fiber composite, titanium, cobalt-chromium, shape memory metal, resorbable polymers, bio-inert metal, bio-inert polymeric materials, and combinations of these materials.

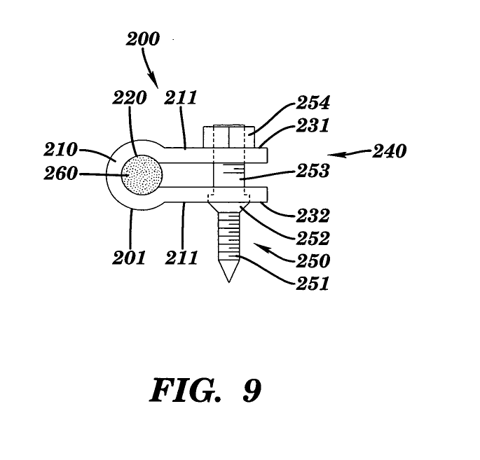

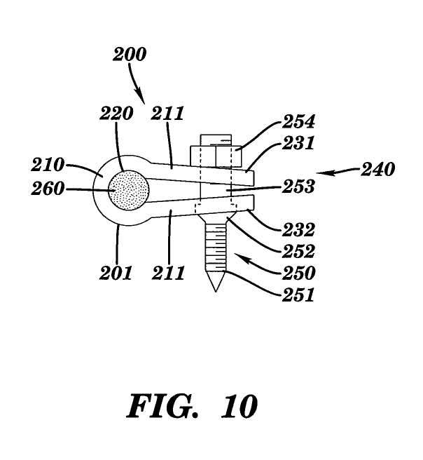

The locking mechanism 240 is generally constructed to include at least one pair of loops 210, the corresponding attached pair of legs 211, a pair of tabs 230 that are comprised of an upper tab 231 and a lower tab 232 and at least one through hole 233 in each tab 230 and collared bone fastener 250 typically including a locking nut 254. As shown in FIG. 8, from each loop 210 may extend a pair of legs 211. The pair of legs 211 typically merge to form a tab 230. The in-series, dual locking mechanism device 10 as seen in FIG. 8 shows the two pairs of legs 211 forming an upper tab 231 and a lower tab 232, although it is understood by one skilled in the art that other configurations may be used. FIG. 9 shows the locking mechanism 240 in the open position, wherein the upper tab 231 and the lower tab 232 are almost parallel relative to each other with a gap between the tabs 230 opposing surfaces. In addition, the collared bone fastener 250 has been inserted through aligned holes 233 in the tabs 230 without the locking nut 254 being operated to its closed position. The open position allows the implant 260 to move freely within the channel 220. The closed position for the locking mechanism 240 as shown in FIG. 10 is typically achieved when the locking nut 254 threadably engages the threaded post 253 causing the upper tab 231 and the lower tab 232 to move in closer proximity resulting in the urging of the pair of loops 210 to pressingly engage the implant 260 in preferably adjacent and in-series locations, substantially along the longitudinal axis of the implant 260.

As shown in FIGS. 8 and 11, the in-series, dual locking mechanism device 10 is preferably secured to a bone by a collared bone fastener 250. At least one through hole 233 for receiving a collared bone fastener 250 is located within the tabs 230. Following the insertion into the tabs 230, the longitudinal axis of the collared bone fastener 250 is oriented approximately normal thereto. As shown in FIG. 13, the collared bone fastener 250 is preferably configured to include three connected members, an externally threaded member 251 for insertion into a bone, a fixed collar member 252, for seating onto the bottom surface of the lower tab 232 and a threaded post member 253 for preferably threadably engaging the locking nut 254. As seen in FIG. 7, the collared bone fastener 250 may be inserted into the tabs 230 passing through the lower tab 232 with the threaded post 252 projecting above the upper tab 231. The in-series, dual locking mechanism device 10 being preferably secured to the collared bone fastener 250 with a locking nut 254.

Though not shown, it is understood to those skilled in the art that the locking mechanism 240 of the alternative embodiment may be structured to include a locking cap 40 that is configured with an external cam surface, the locking cap 40 being inserted into the hole 233 to threadably engage an internal cam surface within the portion of the hole 233 located in the lower tab 232. It is further understood that the bone fastener 30 for this alternative embodiment may be integrally coupled to the under surface of the lower tab 232.

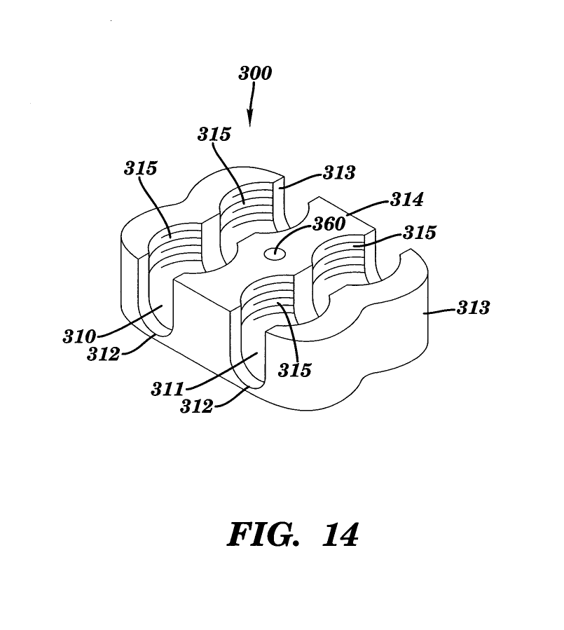

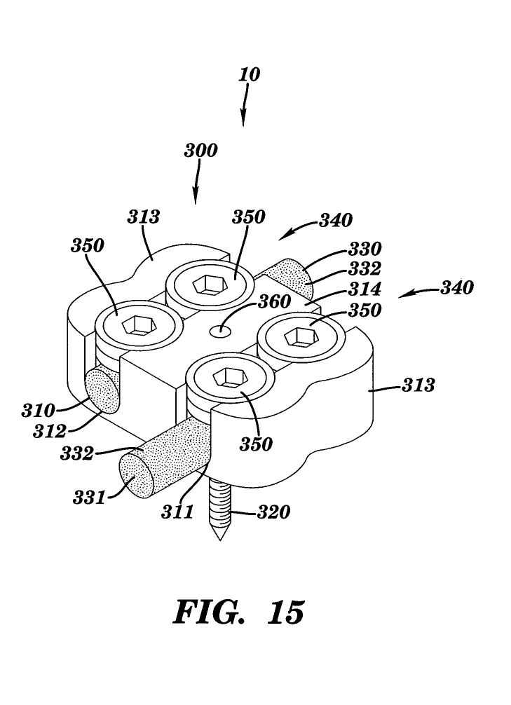

Yet a further embodiment of the in-series, dual locking mechanism device 10 is seen in FIGS. 14-17 that includes two channels 310, 311 that are substantially parallel to each other. FIG. 15 shows the general arrangement of the alternative embodiment of the in-series, dual locking mechanism device 10 that includes a clamp 300, a bone fastener 320, at least two channels 310, 311, at least two implants 330, 331 and a locking mechanism 340. With reference to FIGS. 14 and 15, the in-series, dual locking mechanism device 10 is comprised of a clamp 300 wherein the channels 310, 311 are preferably defined by a floor member 312 and three wall members 313, 314. The wall members 313, 314 being preferably parallel relative to each other with the side wall members 313 and middle wall member 314 being separated by a distance about equal to the width of the floor member 312 respectively. The three wall members 313, 314 project in an upward manner from the floor member 312 to form two U-shaped channels 310, 311 with both being approximately sized to receive an implant 330, 331. The internal sides of the side wall members 313 and both sides of the middle wall member 314 may include internal threads 315 or alternatively internal cam surfaces (Not Shown) preferably to engage a locking cap 350. The wall members 313, 314 preferably include at least two sets of internal threads 315 or alternatively internal cam surfaces. (Not Shown) arranged in-series along the longitudinal axis of the channels 310, 311. As seen in FIG. 14, the two sets of internal threads 315 may be positioned in close proximity or adjacent to each other along the longitudinal axis of their respective channels 310, 311. Typically, as shown in FIG. 14, at least one through hole 360 is located through the middle wall member 314. The hole 360 may receive a bone fastener 320 prior to the insertion of the implants 330, 331. The longitudinal axis of the bone fastener 320 may be at a fixed angle relative to the floor member 312 following insertion into the hole 360 or alternatively, may be allowed to pivot within the hole 360. The hole 360 may be counter bored, counter sunk, slotted, keyed, have a spherical seat or any combination or derivation of these manufacturing techniques, to allow the top portion of the bone fastener 320 to sit within the middle wall member 314. The bone fastener 320 is typically configured as a bone screw (Not Shown), but alternative bone fasteners may be utilized including, but not limited to bone fixation posts (Not Shown), bone staples (Not Shown), hooks (Not Shown), anchors (Not Shown), fixed head screws (Not Shown) and moveable head screws (Not Shown. It is understood to those skilled in the art that the method of attaching the bone fastener 320 to the clamp 300 described herein is for example and that other configurations may be used, including having the bone fastener 320 integrally coupled to the bottom surface of the clamp 300.

As shown in FIGS. 15, 16, 17, the locking mechanism 340 is illustrated as including at least four externally threaded locking caps 350 threadably engaged with the internal threads 315 of the wall members 313, 314, although it is understood by those skilled in the art that other configurations are contemplated, including a locking cap configured to include an external cam surface (Not Shown) that engages with an internal cam surface (Not Shown) located within each wall member 313, 314. In the open position, the threaded locking caps 350 of the locking mechanism 340 initiate engagement with the internal threads 315 of the wall members 313, 314 and allow the implants 330, 331 to move freely within the channels 310, 311. When in the closed position, preferably the threaded locking caps 350 of the locking mechanism 340, are substantially engaged with the internal threads 315 usually resulting in a pressing engagement or compressive force being applied to the top surface 332 of the implants 330, 331. As shown in FIGS. 15, 16 and 17, each implant 330, 331 is locked by at least two locking caps 350 preferably positioned adjacent and in-series to each other, substantially along the longitudinal axis of their respective channels 310, 311. The importance of the configuration of the locking mechanism 340 is discussed above herein.

Preferably, the implants 330, 331 secured within the channels 310, 311 by the in-series, dual locking mechanism device 10 are shaped as elongate and continuous rods. Alternative implants 330, 331 may be secured by the in-series, dual locking mechanism device 10, these include, but are not limited to plates, bars, tethers, cables, elastic structures and dynamic stabilization members. The implants 330, 331 may be comprised of a material selected from the group consisting of stainless steel, carbon fiber composite, titanium, cobalt-chromium, shape memory metal, resorbable polymers, bio-inert metal, bio-inert polymeric materials, and combinations of these materials.

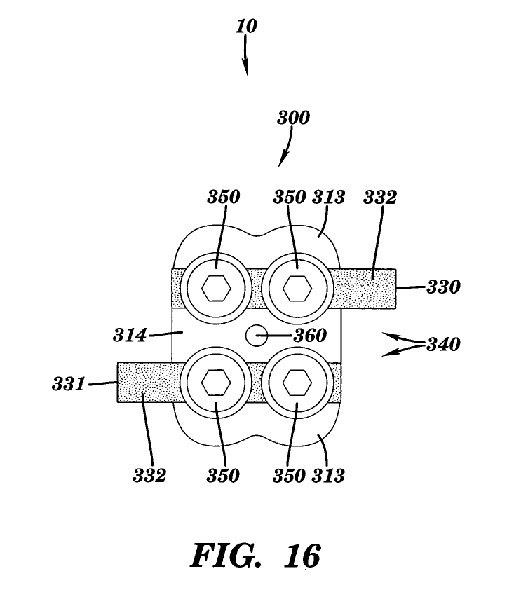

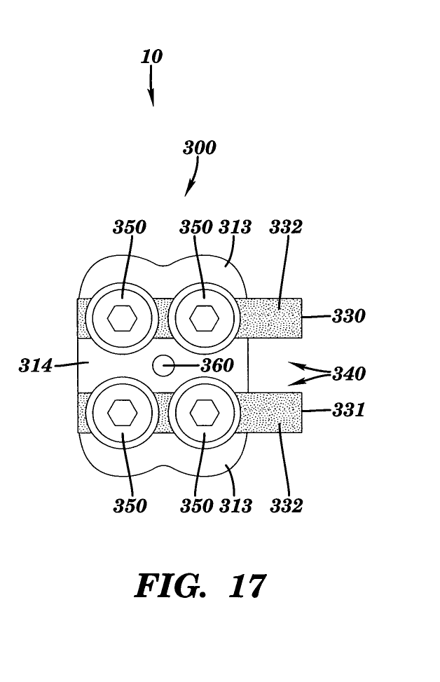

As shown in FIGS. 16 and 17, the implants 330, 331 may be locked in the channels 310, 311 in various construct arrangements, including, but not limited in parallel or in multi-level segmentations. Such arrangements will depend on the structural application that the implants 330, 331 are being utilized to address

The steps of the method to use the in-series, dual locking mechanism device 10 include exposing the bone on which the clamp 20 is to be attached. Depending on the type of bone, a preferred bone fastener 30 is selected from a kit. The next step of the method is to preferably attached the bone fastener 30 to the clamp 20. The next step is to typically drill a pilot hole into at least one bone. The following step is preferably to insert the bone fastener 30 into the pilot hole, fixing the clamp 20 in close approximation to the bone. Following securement of the clamp 20, the last step would be to place the implant 50 within the channel 21. Preferably, the implant 50 is configured as at least one elongate and continuous rod though, alternative implants are contemplated for use in the in-series, dual locking mechanism device 10. These alternative implants include, but are not limited to plates, bars, tethers, cables, elastic structures and dynamic stabilization members. Following the implant placement step, typically the implant 50 is locked within the channel 21 by closing the locking mechanism 60 and thereby pressingly engaging the implant 50 in at least two adjacent and in-series locations along the top surface of the implant 50. It is contemplated that depending upon the embodiment used to lock the implant 50 in place, the locking mechanism 140, 240 may be comprised of at least two locking caps 112 or at least two loops 210, respectively.

Although the preferred embodiments have been depicted and described in detail herein, it will be apparent to those skilled in the relevant art that various modifications, additions and substitutions can be made without departing from its essence and therefore these are to be considered to be within the scope of the following claims.

What is claimed is:

1. A device for securing at least one elongate and continuous orthopaedic implant to a bone and for distributing forces along said implant comprising:
a clamp having a channel for receiving the implant therein;
a locking mechanism operatively associated with the clamp for securing said implant in the clamp, said locking mechanism being operable between an open position and a closed position and the locking mechanism being configured to cause pressing engagement between the clamp and the implant received therein in at least two adjacent and in-series locations on the implant when operated to its closed position; and
a bone fastener projecting from the clamp and configured for affixing the clamp to a bone.

2. The device of claim 1 in combination with the implant.

3. The device of claim 1 wherein the channel is defined by a floor member and a pair of wall members connected thereto so as to form a U-shaped channel.

4. The device of claim 3 wherein the wall members are configured to receive a locking mechanism.

5. The device of claim 4 wherein the wall members are configured to include an internal cam surface in at least two locations, and the locking mechanism is comprised of at least two corresponding locking caps having external cam surfaces.

6. The device of claim 4 wherein the wall members are internally threaded in at least two locations, and the locking mechanism is comprised of at least two corresponding locking caps having external threads.

7. The device of claim 3 wherein the floor member includes at least one hole configured to receive a bone fastener, the bone fastener having a longitudinal axis wherein the longitudinal axis extends in a direction substantially orthogonal to the floor member.

8. The device of claim 3 wherein the floor member includes at least one hole configured to receive a bone fastener, the bone fastener having a longitudinal axis wherein the longitudinal axis is angled to the floor member.

9. The device of claim 2 wherein the implant is a rod.

10. The device of claim 1 wherein the bone fastener is a screw.

11. The device of claim 1 wherein the bone fastener is a post.

12. The device of claim 1 wherein the bone fastener is a staple.

13. The device of claim 1 wherein the bone fastener is a hook.

14. The device of claim 1 wherein the bone fastener is an anchor.

15. The device of claim 1 wherein the bone is a vertebral body.

16. The device of claim 1 wherein the clamp is comprised of a cylinder through which said channel passes longitudinally.

17. The device of claim 16 wherein the cylinder is configured so as to include at least two holes adjacent and in-series relative to each other and passing from an exterior surface of the cylinder into its channel, the holes being configured to include an internal cam surface, and being positioned to receive the locking mechanism.

18. The device of claim 17 wherein the locking mechanism includes at least two corresponding locking caps configured to include an external cam surface.

19. The device of claim 16 wherein the cylinder is configured so as to include at least two internally threaded holes adjacent and in-series relative to each other and passing from an exterior surface of the cylinder into its channel, and the holes being positioned to receive the locking mechanism.

20. The device of claim 19 wherein the locking mechanism includes at least two corresponding externally threaded locking caps.

21. The device of claim 16 wherein the clamp is further comprised of an offset flange rigidly fixed to the cylinder.

22. The device of claim 21 wherein the offset flange includes at least one hole for receiving a bone fastener.

23. The device of claim 21 wherein a bone fastener is integrally fixed to the offset flange.

24. The device of claim 1 wherein the clamp is comprised of a springy material in the form of a bracket having at least a pair of adjacent and in-series loops defining the channel, each loop having a pair of legs.

25. The device of claim 24 wherein adjacent ones of each pair of legs are joined by a tab so as to form at least one pair of opposing and normally spaced apart tabs.

26. The device of claim 25 wherein the locking mechanism is effective to move the tabs together thereby urging the loops into pressing engagement with the implant.

27. The device of claim 26 wherein the tabs are formed with aligned holes.

28. The device of claim 27 wherein the locking mechanism is comprised of a threaded post attached to the bone fastener and configured to pass through the aligned holes and a correspondingly threaded nut.

29. The device of claim 27 wherein the locking mechanism is comprised of a locking cap having an external cam surface configured to pass through the aligned holes and engage an internal cam surface in an aligned hole.

30. The device of claim 1 wherein the clamp is comprised of a first channel for the purpose of receiving a first implant therein and a second channel for the purpose of receiving a second implant therein, the first channel being substantially parallel and adjacent to the second channel, the first channel and the second channel each having a locking mechanism.

31. A method for securing an implant to a bone comprising the steps of:
providing a device comprised of a clamp having a channel therein for receiving an implant, a locking mechanism being operatively associated with said clamp for securing the implant in the clamp, said locking mechanism being configured when closed to cause pressing engagement between the clamp and the received implant in at least two adjacent and in-series locations, and a bone fastener attachable to said clamp, said bone fastener being configured to affix said clamp to a bone;
attaching the bone fastener to said clamp;
drilling a pilot hole into the bone;
inserting said bone fastener into said pilot hole in the bone so as to secure the clamp to the bone;
placing an implant into said channel; and
locking said implant into said clamp.

32. The method of claim 31 wherein the locking step includes the step of closing the locking mechanism.

33. A kit for securing at least one elongate and continuous orthopaedic implant to a bone, the kit comprising:
a clamp having at least one channel adapted to receive the implant;
a locking mechanism adapted to be operatively connected to the clamp for causing pressing engagement between the implant and the clamp in at least two adjacent and in-series locations; and
at least one bone fastener attachable to the clamp and configured to fixate the clamp to a bone.

34. The kit of claim 33 wherein:
said bone fastener is a screw.

35. The kit of claim 33 wherein:
said bone fastener is a post.

36. The kit of claim 33 wherein:
said bone fastener is a staple.

37. The kit of claim 33 wherein said bone fastener is a hook.

38. The kit of claim 33 wherein said bone fastener is an anchor.

39. The kit of claim 33 wherein:
said implant is a rod.

40. The kit of claim 33 wherein:
said implant is a flexible cable.
